(19) United States
(12) Patent Application Publication
(10) Pub. No.: US 2007/0119472 A1
(43) Pub. Date: May 31, 2007

(54) Labeled hair color brush

(76) Inventor: Rosemary S. White West, Fortuna, CA (US)

Correspondence Address: ROSEMARY SHARON WHITE WEST, P.O. BOX 54, FORTUNA, CA, 95640, US

(21) Appl. No.: 11/287,431

(22) Filed: Nov. 28, 2005

(51) Int. Cl. A61K 8/18

(52) U.S. Cl. 132/208

(57) ABSTRACT

A hair coloring kit, including a plurality of brush applicators, in which the brushes,are colored and numbered to correspond with the color and formulation of the hair color or bleach being applied to the hair during a hair coloring process. Said brush applicators vary in shape and size to allow for larger or smaller areas of hair to be covered with the hair color or bleach.

### FIELD OF THE INVENTION

Hair color brush applicators labeled and colored to directly correspond with the color and formulas of hair color or bleaches being used during a hair color service in a hair salon.

### BACKGROUND OF THE INVENTION

Many hair color services require more than one color to be used during the application, or more than one formula for a variety of reasons. Different colors such as for highlights and lowlights require multiple amounts of formulas. Often a hairstylist will use the same color on the hair but will use a variety of levels of hydrogen peroxide. An example of this is darker hair at the scalp, with lighter ends. If the hairstylist is to make the scalp hair as light as the ends, the need for a higher level of peroxide exists. If the hairstylist desires to brighten the ends without incurring damage to those ends, there is a need for a much lower level of peroxide and yet the same color would be used. Often there is the need for at least three formulations of hair color or bleach to be used on the same person during a color service.

There is a need for a simple, fast and inexpensive way for the hairstylist to determine which formula is which. Often hair color and bleach formulas look the same in their containers, especially after the hydrogen peroxide is added, causing the formula to oxidize and resemble the other formulas being used. For the hair to be in optimum condition following the color service, the application of the color or bleach should be as fast and precise as possible.

The present invention helps safeguard the hair by identifying each formula according to the applicator brush inserted in each. Thus, fewer mistakes are made by the hairstylist.

The present invention has a variety of brush applicators in various sizes and shapes. The various shapes allow for efficiency during a hair color or bleach service by fitting the amount of hair to be colored or bleached. Many people desire to have their eyebrows colored and a smaller brush applicator is indicated to keep the formula from contact with the skin. A smaller brush applicator is also easier to use while applying hair color or bleach to small streaks on the hair, as is done for highlights and lowlights.

### SUMMARY OF THE INVENTION

The present invention relates to a kit of hair color brush applicators. Said brush applicators are varied in size, to accommodate a given amount of hair to be colored or bleached. A small area of hair to be colored or bleached, such as eyebrow hairs would be more efficiently covered by the color formula with a small brush, thereby keeping the color mixture from the skin of the client. A hairstylist wishing to apply small streaks of color or bleach highlights or lowlights would also benefit from the smaller brush applicator as the formula is easily applied to a smaller area by using a smaller brush, thereby avoiding contact with hair that is not to be covered with the hair color mixture.

The present invention has a variety of colors that are the corresponding colors of the hair color or bleach formula to be applied to the hair. It is an object of the invention to help the hairstylist determine the formula in each color container by observing which brush is in the container. For example, a dark red brush applicator will indicate that the finished product of that formula in which the brush is immersed, will be dark red. As hair color can look similar as it oxidizes in the container, it is easy to lose track of each one's tonal value.

It is an object of the invention to allow the hairstylist to ascertain the formulas in each application container by using labeled brush applicators that are used to paint the hair color or bleach on the hair. Each labeled brush indicates the level of hydrogen peroxide in that particular formula. By observing the color of the brush and the label, the hairstylist will be able to tell the difference between formulas when a plurality of formulas ate being used.

### BRIEF DESCRIPTION OF THE DRAWINGS

FIG. 1 is an illustration of a single blonde tint, with three levels of hydrogen peroxide. Level 10 strength is the lowest strength shown. Level 30 is the highest strength shown.

FIG. 2 illustrates tonal values ranging from blonde to brown. Levels of peroxide are indicated by the numbers on the brush applicator handles.

FIG. 3 indicates a variety of red colors of brush applicators, to be used with similar colored hair color formulas. The label on each brush indicates 40 volume peroxide for that particular formula.

FIG. 4 illustrates a small brush applicator.

### DETAILED DESCRIPTION OF THE INVENTION

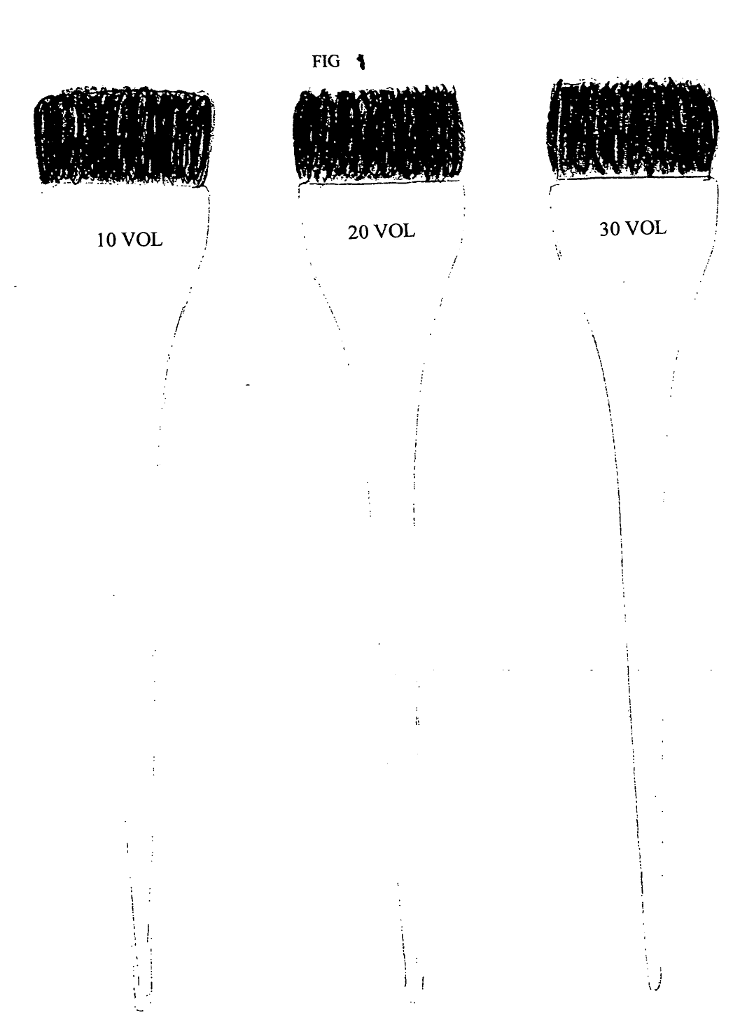

FIG. 1 illustrates several brush applicators that are a blonde in color and have labels. Each brush represents a color formula. Represented are three strengths of hair color with each having the same color or tonal value.

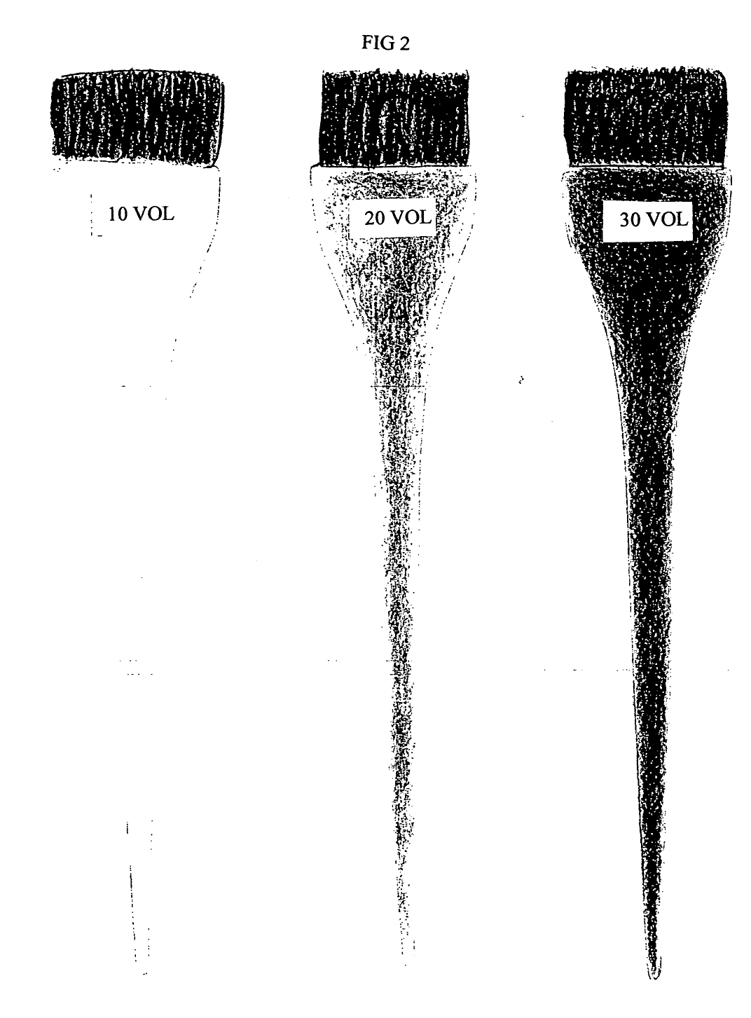

FIG. 2 illustrates levels of hair color, by the color of each brush applicator. A formula for a blonde shade may require the first brush shown. A brush of the darkest level may be used with a hair color that would be that shade. The label on each brush indicates the level of hydrogen peroxide included in the hair color formula or bleach formula in which the brush is immersed.

Hair stylists mix hair color and bleach formulas with hydrogen peroxide. These are mixed in unmarked containers and are usually applied with plastic applicator bottles or applicator brushes. These are also unlabeled. As hair color or bleach is mixed with peroxide, it begins to oxidize. Oxidization causes the formulas to lose their original color, temporarily, and to resemble other hair colors and bleaches. This is a problem if more than one formula is to be used during the same color service. Often several formulas are used at a time during a color service in a hair salon. It can be confusing to the hairstylist, if the oxidized formulas resemble each other. The present invention ends this confusion, with each formula having its own container and labeled, color corresponding brush.

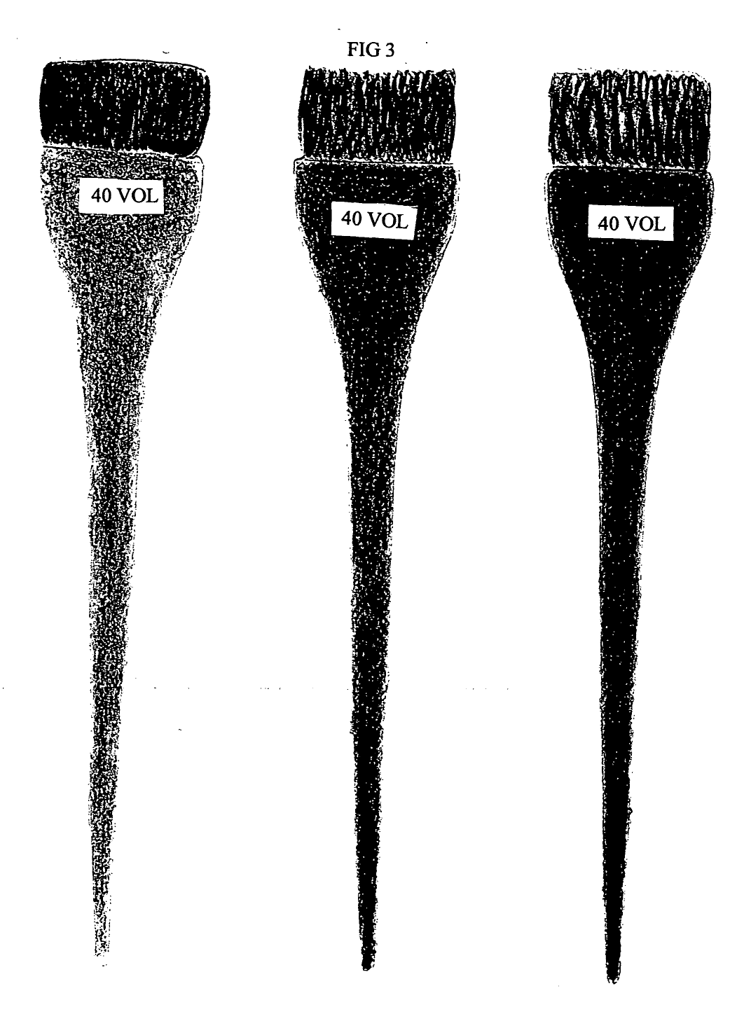

FIG. 3 illustrates three different shades of red, all of which are labeled 40 VOL to indicate the level of peroxide to accompany the red in said formula.

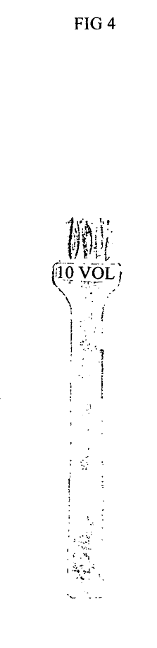

FIG. 4 illustrates a small brush applicator that is labeled with peroxide levels and colored to match the shade of color in said formula. Traditionally, hair color brushes are large. They usually have a six inch handle or longer and the bristles are at least an inch in length. This is too big for smaller color applications, such as with coloring eyebrows. A smaller brush is easier to use and is less messy. It is easier to keep the formula off the skin of the client, making it safer for the client. The color and label on the smaller brush indicate the formula being used. The smaller brush makes small highlights, lowlights and streaks easier to accomplish without getting hair color or bleach on the rest of the hair.

With the present invention, the hairstylist can avoid the confusion that accompanies a plurality of formulas and apply the formulas faster. Hair color should be applied quickly to avoid uneven processing and damage to the hair. The present invention enables the stylist to know what is in each container, making the application of the formula faster and safer for the client's hair. If the wrong hair color or bleach formula is used, it can cause damage to the hair. If a client has an inch of dark hair at the scalp, and all the rest of the hair is a light blonde, it usually takes a high volume of peroxide to lift the scalp hair color to the shade of blonde on the rest of the hair. If the same client were to have the ends of said hair lightened just one shade or level, this would require a lower volume of peroxide, because the hair is already lighter than the scalp hair. A problem exists when unmarked containers resemble one another. If the high volume peroxide were inadvertently applied to the already lighter ends of the hair, there would be damage to that hair. Likewise, if the lower volume peroxide were applied to the scalp hairs, those would be under processed and not as light as the rest of the hair.

The present invention is made of a handle of a hard, durable material, such as a plastic. The bristles are of a soft and flexible material such as nylon. The brush applicators are light weight and inexpensive. They take up very little room and are durable.

What is claimed is:

1. A hair coloring kit comprised of a plurality of brush applicators having the color of the hair dye or bleach that will be applied to the hair during a hair color service and being comprised of a hard and durable material, such as plastic for the handles and nylon for the bristles, but not limited to plastic or nylon materials.

2. Brush applicators have a variety of sizes and shapes and colors to correspond directly to the formula, color and size of the hair color service.

3. Brush Applicators are labeled to indicate the level or amount of hydrogen peroxide in the formula, in which the brush is used.

4. A method of coloring or bleaching hair with a plurality of formulas and a plurality of techniques to be comprised of: mixing individual hair color formations in a container and inserting matching colored brush applicators in each formula, with labels on each brush applicator that indicate the formula in the corresponding container; with said brush applicators being a variety of sizes and shapes to better accommodate and fit the actual amount of hair to be colored or bleached.
